(19) United States
(12) Patent Application Publication
(10) Pub. No.: US 2026/0018248 A1
(43) Pub. Date: Jan. 15, 2026

(54) Systems and Methods for Nucleic Acid Data Tokenization

(71) Applicant: The Board of Trustees of the Leland Stanford Junior University, Stanford, CA (US)

(72) Inventor: Julia Salzman, Palo Alto, CA (US)

(73) Assignee: The Board of Trustees of the Leland Stanford Junior University, Stanford, CA (US)

(21) Appl. No.: 19/266,056

(22) Filed: Jul. 10, 2025

Related U.S. Application Data
(60) Provisional application No. 63/670,074, filed on Jul. 11, 2024.

(51) Int. Cl. G16B 30/10; G16B 40/20; G16B 50/50

(52) U.S. Cl. CPC G16B 30/10; G16B 40/20; G16B 50/50

(57) ABSTRACT

Systems and methods for nucleic acid data tokenization in accordance with embodiments of the invention are illustrated. One embodiment includes a method for tokenizing genetic sequence data, comprising obtaining genetic sequence data, extracting k-mers from the genetic sequence data as a plurality of k-mer anchors, appending at least one most abundant k-mer target to each k-mer anchor, and generating tokens based on the appended k-mer anchors and targets. In a further embodiment, extracting k-mers from the genetic sequence data includes using a Statistically Primary alignment Agnostic Sequence Homing (SPLASH) technique. In still another embodiment, the method further includes steps for appending a count for each appended k-mer target to the k-mer anchor.

### CROSS-REFERENCE TO RELATED APPLICATIONS

The current application claims the benefit of and priority under 35 U.S.C. § 119(e) to U.S. Provisional Patent Application No. 63/670,074 entitled “Methods for Biological Data Tokenization” filed Jul. 11, 2024. The disclosure of U.S. Provisional Patent Application No. 63/670,074 is hereby incorporated by reference in its entirety for all purposes.

### GOVERNMENT FUNDING STATEMENT

This invention was made with Government support under contract GM139517 awarded by the National Institutes of Health. The Government has certain rights in the invention.

### FIELD OF INVENTION

The present invention generally relates to methods for tokenizing unpredictable biological data to improve signal compression.

### BACKGROUND

Artificial intelligence (AI) models are increasingly becoming a critical technology in many fields. Transformer models such as ChatGPT, and other large language models are rapidly evolving in complexity. However, despite more sophisticated model architectures, fundamentally AI models are reliant upon training with large quantities of data. At least in the text domain, ingesting these data involves a processes referred to as tokenization, whereby data is cut up into tokens that can be mapped (or “embedded”) into a vector space. Similarly, post-training, user inputs are also tokenized when provided to the model. As all inputs throughout the life cycle of a text AI model go through tokenization, the method of tokenization can have significant impact on the performance of the model.

SPLASH (Statistically Primary alignment Agnostic Sequence Homing) is a genomics workflow that directly analyzes raw sequencing data to detect sample-specific sequence variation. The fundamental concept of SPLASH is anchors and targets. An “anchor” is any particular k-mer of sequence in a read. Every k-mer a fixed offset downstream is called a “target”. Targets are always defined relative to an anchor, and a given anchor may have multiple associated targets. SPLASH is described in Chaung et al., “SPLASH: a statistical, reference-free genomic algorithm unifies biological discovery”, Cell, Volume 186, Issue 25, 5440-5456.e26.

### SUMMARY

Systems and methods for nucleic acid data tokenization in accordance with embodiments of the invention are illustrated. One embodiment includes a method for tokenizing genetic sequence data, comprising obtaining genetic sequence data, extracting k-mers from the genetic sequence data as a plurality of k-mer anchors, appending at least one most abundant k-mer target to each k-mer anchor, and generating tokens based on the appended k-mer anchors and targets.

In a further embodiment, extracting k-mers from the genetic sequence data includes using a Statistically Primary alignment Agnostic Sequence Homing (SPLASH) technique.

In still another embodiment, the method further includes steps for appending a count for each appended k-mer target to the k-mer anchor.

In a still further embodiment, generating tokens includes replacing absent sequences in a sample with a special token.

In yet another embodiment, the special token is ‘N’.

In a yet further embodiment, the method further includes steps for filtering the k-mer anchors based on entropy and effect size thresholds.

In another additional embodiment, the method further includes steps for checking the filtered k-mer anchors against contaminant and positive lookup tables to identify and exclude certain sequences.

One embodiment includes a system for analyzing nucleic acid sequences, comprising a processor, and a memory storing instructions that, when executed by the processor, cause the system to receive genetic sequence data, extract k-mers from the genetic sequence data as a plurality of k-mer anchors, append at least one most abundant k-mer target to each k-mer anchor, and generate tokens based on the appended k-mer anchors and targets.

In a further additional embodiment, extracting k-mers from the genetic sequence data includes using a Statistically Primary alignment Agnostic Sequence Homing (SPLASH) technique.

In another embodiment again, the instructions further cause the system to append a count for each appended k-mer target to the k-mer anchor.

In a further embodiment again, generating tokens includes replacing absent sequences in a sample with a special token.

In still yet another embodiment, the special token is ‘N’.

In a still yet further embodiment, the instructions further cause the system to filter the k-mer anchors based on entropy and effect size thresholds.

In still another additional embodiment, the instructions further cause the system to check the filtered k-mer anchors against contaminant and positive lookup tables to identify and exclude certain sequences.

One embodiment includes a non-transitory computer-readable storage medium storing instructions that, when executed by a processor, cause the processor to perform operations comprising obtaining genetic sequence data, extracting k-mers from the genetic sequence data as a plurality of k-mer anchors, appending at least one most abundant k-mer target to each k-mer anchor, and generating tokens based on the appended k-mer anchors and targets.

In a still further additional embodiment, extracting k-mers from the genetic sequence data includes using a Statistically Primary alignment Agnostic Sequence Homing (SPLASH) technique.

In still another embodiment again, the operations further includes appending a count for each appended k-mer target to the k-mer anchor.

In a still further embodiment again, generating tokens includes replacing absent sequences in a sample with a special token.

In yet another additional embodiment, the special token is ‘N’.

In a yet further additional embodiment, the operations further includes filtering the k-mer anchors based on entropy and effect size thresholds, and checking the filtered k-mer anchors against contaminant and positive lookup tables to identify and exclude certain sequences.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF FIGURES

The description and claims will be more fully understood with reference to the following figures and data graphs, which are presented as exemplary embodiments of the invention and should not be construed as a complete recitation of the scope of the invention.

FIG. 1 illustrates a block diagram of a nucleic acid analysis system, according to aspects of the present disclosure.

FIG. 2 illustrates a block diagram of a nucleic acid analyzer, according to an embodiment.

FIG. 3 illustrates a flowchart of a process for generating tokens from genetic sequence data, in accordance with example embodiments.

FIG. 4 illustrates a flowchart of another process for generating tokens from genetic sequence data, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

SPLASH (Statistically Primary alignment Agnostic Sequence Homing) is utilized to generate k-mer anchors and targets from genetic sequence data. In this process, an anchor is defined as any particular k-mer of sequence in a read, while a target is every k-mer a fixed offset downstream from the anchor. This approach allows for the identification of sample-specific sequence variations without the need for a reference genome.

The k-mer anchors and targets generated through SPLASH are converted into tokens through a process of appending the most abundant k-mer target(s) for each k-mer anchor. This tokenization method involves extracting k-mers from the genetic sequence data as a plurality of k-mer anchors, identifying the most frequent k-mer targets associated with each anchor in each sample, and combining the anchor-target pairs to form tokens. In many embodiments, the most frequent k2-mer targets are identified. The length k2 may be different than the length k of the anchor. In various embodiments, other sequences than targets downstream or upstream of an anchor are used, and at any gap length. For example, the 1^{st}, 4^{th}, and 6^{th} nucleotides up and downstream can be used. In addition to a single target per anchor, multiple targets per anchor may be used as tokens and multiple k-mer lengths can be used. Samples can be any fastq file or a sample determined by a barcode, for example.

This approach to tokenizing genetic sequence data offers several advantages. Firstly, it allows for signal compression by reducing the dimensionality of the genetic data while retaining important sequence relationships. The anchor-target pairs capture local sequence context more efficiently than individual nucleotides or fixed-length k-mers alone. In many embodiments, the tokens are translations of nucleotides, and occur as amino acids.

Secondly, this method improves signal resolution by preserving information about both the anchor sequences and their associated downstream targets. This contextual information enables more nuanced analysis of genetic variations and their potential functional implications.

Finally, the tokenization approach based on SPLASH-derived anchor-target pairs accelerates transformer training and fitting for sequence analysis. In many embodiments, the transformer is a large language model. By providing a more compact and information-rich representation of genetic data, or amino-acid representations, these tokens enable more efficient processing and learning of sequence patterns by AI models. This leads to improved performance in tasks such as variant calling, gene expression prediction, or functional genomics analyses.

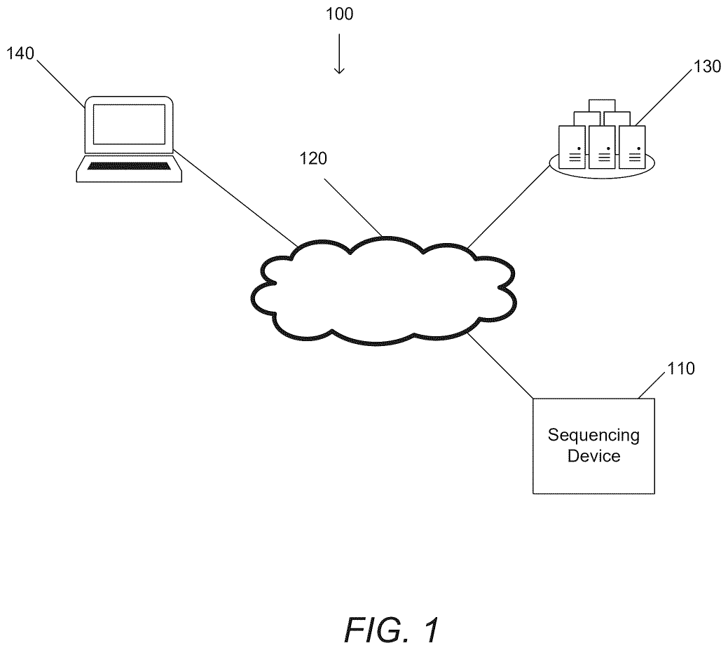

FIG. 1 illustrates a nucleic acid analysis system 100. The nucleic acid analysis system 100 includes a sequencing device 110, a network 120, a nucleic acid analyzer 130, and a display device 140. In some embodiments, the sequencing device is configured to determine DNA and RNA sequences from biological samples. The sequencing device generates genetic sequence data from the biological samples. The network enables communication between components of the nucleic acid analysis system. In various embodiments, the network includes the Internet. The network represents multiple interconnected networks in certain embodiments.

The nucleic acid analyzer 130 receives genetic sequence data from the sequencing device 110 through the network 120. In various embodiments, the nucleic acid analyzer 130 processes and analyzes the received genetic sequence data. The display device 140 is connected to the nucleic acid analyzer 130. In some embodiments, the display device 140 presents analysis results generated by the nucleic acid analyzer 130. The components of the nucleic acid analysis system 100 work together to process genetic sequence data. For example, the sequencing device 110 generates genetic sequence data and transmits the data through the network 120 to the nucleic acid analyzer 130. The nucleic acid analyzer 130 then processes the received data and outputs results to the display device 140 for presentation. As is readily appreciated, FIG. 1 represents a particular architecture, however various processes are performed using only nucleic acid analyzers in accordance with various embodiments of the invention.

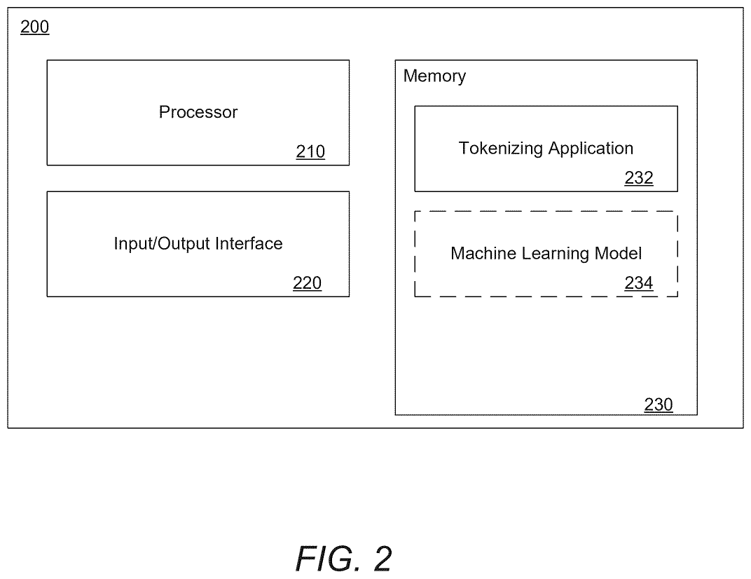

FIG. 2 illustrates a nucleic acid analyzer 200. The nucleic acid analyzer 200 includes a processor 210, an input/output device 220, and memory 230. In various embodiments, the processor executes instructions and processes data for tokenizing nucleic acid sequence data. The input/output device enables communication with external devices and systems, such as sequencing devices and display devices. In many embodiments, input/output devices allow for input of nucleic acid sequence data and output of analysis results.

The memory 230 contains a tokenizing application 232. In some embodiments, the memory 230 also includes a machine learning model 234, though the machine learning model 234 is not always stored in memory. In many embodiments, the machine learning model is a transformer model, but any machine learning model that utilizes tokens can be used as appropriate to the requirements of specific applications of embodiments of the invention. The tokenizing application 232 processes nucleic acid sequence data to generate tokens based on k-mer analysis. In various embodiments, the tokenizing application utilizes SPLASH to generate k-mer anchors and targets, which in turn are used to generate tokens as described below. The machine learning model 234, when present, utilizes the generated tokens for analyzing and processing nucleic acid sequence data.

The components of the nucleic acid analyzer 200 are interconnected to enable processing of nucleic acid sequence data. In many embodiments, the processor 210 coordinates operations between the input/output device 220 and the tokenizing application 232 stored in the memory 230. The nucleic acid analyzer 200 receives genetic sequence data from the sequencing device 110 through the network 120, processes the data using the tokenizing application 232, and outputs results through the input/output device 220 to the display device 140.

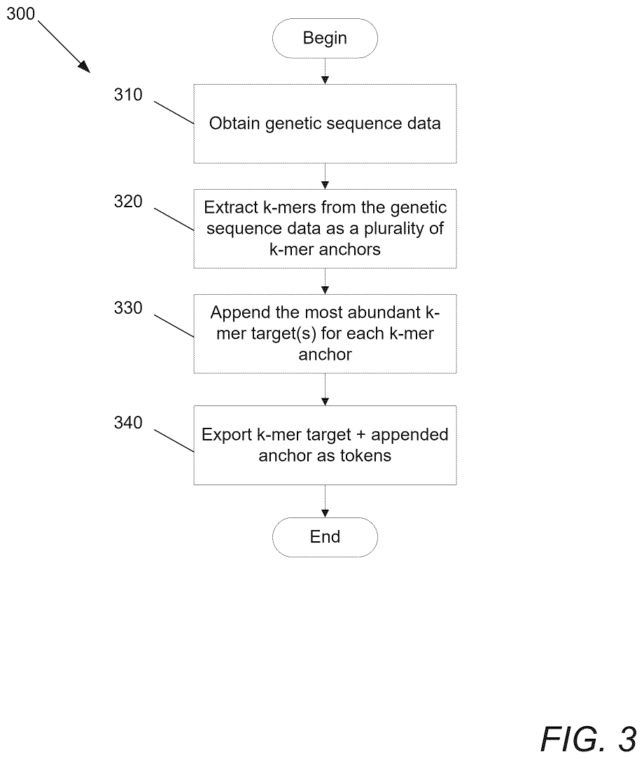

FIG. 3 illustrates a process for generating tokens 300 from genetic sequence data. The process for generating tokens 300 includes multiple steps for processing and analyzing genetic sequence data to create tokens for use in machine learning model or other analysis techniques.

The process for generating tokens 300 begins with obtaining genetic sequence data 310. In some embodiments, the genetic sequence data is received from the sequencing device 110 through the network 120. The genetic sequence data includes DNA or RNA sequences from biological samples.

After obtaining the genetic sequence data, the process for generating tokens 300 proceeds to extract k-mers from the genetic sequence data as a plurality of k-mer anchors 320. In various embodiments, the k-mers are extracted using SPLASH. The SPLASH technique identifies k-mer anchors and associated target sequences within the genetic data.

The process for generating tokens 300 then moves to append the most abundant k-mer target(s) for each k-mer anchor 330. In many embodiments, multiple most abundant k-mer targets are appended to each k-mer anchor, rather than just the single most abundant target. The tokenizing application 232 performs this appending step.

In various embodiments, a count for each appended k-mer target is appended to the k-mer anchor. This count information provides additional context about the frequency of specific target sequences associated with each anchor.

The process for generating tokens 300 includes filtering steps not explicitly shown in FIG. 3. For example, anchors are filtered based on entropy and effect size thresholds. In some embodiments, anchors are checked against contaminant and positive lookup tables to identify and potentially exclude certain sequences.

Following the appending step, the process for generating tokens 300 concludes with exporting k-mer target+appended anchor as tokens 340. The exported tokens are used for further analysis or as input for machine learning models.

In various embodiments, the process for generating tokens 300 includes additional steps not explicitly shown in FIG. 3. For example, absent sequences in a sample are replaced with special tokens such as ‘N’. This replacement helps maintain consistent token length and provides information about missing or uncertain sequences.

The process for generating tokens 300 generates multiple input formats. In many embodiments, these formats include padded and unpadded versions, as well as anchor-target, anchor-only, and target-only representations. These different formats provide flexibility for various analysis techniques or model architectures.

The tokenizing application 232 performs hierarchical sorting of anchors based on multiple statistics. This sorting helps prioritize certain anchors or sequences for analysis based on their statistical properties or biological relevance.

In various embodiments, the process for generating tokens 300 involves consolidating SATC (Sequence Anchor Target Count) files from multiple datasets. This consolidation allows for more comprehensive analysis across diverse genetic datasets.

For example, in many embodiments, a method for consolidating SATC files involves hierarchical merging based on shared anchor sequences. In this approach, SATC files from different datasets may be first grouped by common anchor sequences. For each group, the target sequences and their associated counts may be combined, with counts being summed across datasets for identical target sequences. This hierarchical structure may allow for efficient comparison of anchor-target relationships across multiple datasets while preserving dataset-specific information. The merged SATC files may then sorted based on aggregate target counts, potentially revealing conserved or variable regions across different samples or experimental conditions. This consolidation method may facilitate the identification of consistent anchor-target pairs across diverse datasets, which may be particularly useful for discovering conserved genetic elements or common variations in large-scale genomic studies.

The process for generating tokens 300 is executed by the processor 210 of the nucleic acid analyzer 200. The tokenizing application 232 stored in the memory 230 provides instructions for carrying out the various steps of the process. The resulting tokens are used by the machine learning model 234 for further analysis of the genetic sequence data.

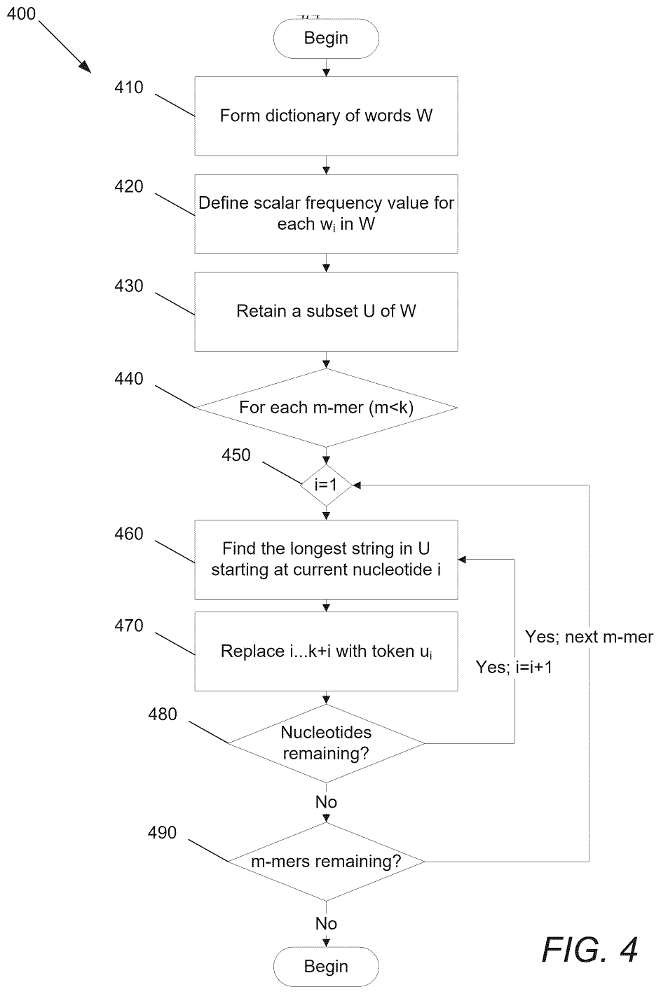

FIG. 4 illustrates a process for generating tokens 400 from genetic sequence data. The process for generating tokens 400 is an expansion of the step of appending the most abundant k-mer target(s) for each k-mer anchor 330 in the process for generating tokens 300.

The process for generating tokens 400 begins with forming a dictionary of words W 410. In various embodiments, the dictionary of words W includes k-mer anchors extracted from the genetic sequence data. The tokenizing application 232 performs this step using compression techniques such as Lempel-Ziv compression on the k-mers.

After forming the dictionary, the process for generating tokens 400 proceeds to define a scalar frequency value for each word wi in W 420. In many embodiments, this scalar frequency value represents the occurrence frequency of each k-mer target associated with a particular k-mer anchor. The processor 210 calculates these frequency values based on the genetic sequence data.

The process for generating tokens 400 then moves to retain a subset U of W 430. In some embodiments, this subset U includes the most frequent k-mer targets for each k-mer anchor. In a variety of embodiments, U includes a random sampling of k-mer targets. The tokenizing application 232 selects this subset based on predefined criteria or thresholds.

Following the retention of subset U, the process for generating tokens 400 proceeds to process each m-mer, where m is less than k 440. This step involves multiple sub-steps for processing the genetic sequence data. For each m-mer, the process sets an index i to 1 450. The index i represents the starting position for processing within the current m-mer. The process then finds the longest string in U starting at the current nucleotide i 460. In various embodiments, this step involves comparing the sequence starting at position i with the k-mer targets in subset U to find the longest match. After finding the longest matching string, the process replaces i through k₊ᵢ with token uᵢ 470. This replacement effectively tokenizes the matched portion of the sequence.

The process for generating tokens 400 includes decision points to control the flow of processing. At step 480, the process checks if nucleotides remain in the current m-mer. If nucleotides remain, the process returns to step 450 to continue processing. If no nucleotides remain, the process advances to step 490. This flow enables recursive generation of the tokens. However, other methods may be used such as a divide and conquer approach.

At step 490, the process checks if m-mers remain to be processed. If m-mers remain, the process returns to step 440 to process the next m-mer. If no m-mers remain, the process ends.

In various embodiments, the process for generating tokens 400 incorporates additional techniques for processing and analyzing the genetic sequence data. For example, the tokenizing application 232 performs multiple-sequence alignment of k-mers to define clusters. These clusters are based on members having another member within a specified Hamming or Levenshtein distance.

The process for generating tokens 400 also includes non-random ordering of k-mers. In many embodiments, this ordering is based on algorithms such as Cholesky decomposition or Singular Value Decomposition (SVD). The processor 210 performs these calculations to determine the optimal ordering of k-mers.

In various embodiments, the process for generating tokens 400 encodes k-mers with a graph structure. This graph structure represents relationships between different k-mers or k-mer clusters, potentially capturing more complex sequence patterns.

The tokenizing application 232 also incorporates edit distance representations for tokens. In some embodiments, these representations are based on Hamming distance, Levenshtein distance, or other biologically meaningful distance metrics. These distance-based representations provide additional context for analyzing sequence similarities and differences.

The process for generating tokens 400 is executed by the processor 210 of the nucleic acid analyzer 200. The tokenizing application 232 stored in the memory 230 provides instructions for carrying out the various steps of the process. The resulting tokens are used by the machine learning model 234 for further analysis of the genetic sequence data.

The tokens generated through the processes described above may be utilized as input data for training machine learning models. In some embodiments, these tokens, which capture important sequence relationships and contextual information from genetic data, can be fed into the model during the training phase. The tokenization approach based on k-mer anchors and targets may provide a more compact and information-rich representation of genetic sequences compared to traditional tokenization methods.

Using these specific tokens in language model training may offer several potential benefits. The tokens may encode biological context and sequence patterns in a way that is more readily interpretable by the model. This could lead to improved performance in tasks related to genetic sequence analysis, such as variant calling or gene expression prediction. Additionally, the hierarchical nature of the token generation process may allow the model to learn multi-scale representations of genetic data, potentially enabling more nuanced understanding of genomic structures.

The training approach utilizing these tokens may result in language models with enhanced capabilities in various genomic applications. For instance, models trained on these tokens may exhibit improved accuracy in predicting functional effects of genetic variations or identifying conserved regulatory elements across species or prediction of the binding of a drug or resistance to that drug. In some cases, the models may develop a more sophisticated understanding of the relationship between genetic sequences and phenotypic traits, potentially aiding in areas such as personalized medicine or crop improvement.

Furthermore, the flexibility in token formats (e.g., padded, unpadded, anchor-only, target-only) described above may allow for experimentation with different input representations during model training. This versatility may enable researchers to optimize model architectures and training strategies for specific genomic analysis tasks.

In various embodiments, the consolidation of files from multiple datasets, as described above, may allow for training on diverse genetic datasets. This approach may lead to more robust and generalizable language models capable of analyzing genetic sequences from a wide range of organisms or experimental conditions.

The incorporation of additional information, such as frequency counts and distance-based representations, into the tokens may provide the language model with valuable contextual cues during training. This enriched input data may enable the model to capture subtle patterns and relationships within genetic sequences that might be missed by more simplistic tokenization approaches.

A number of implementations have been described. Nevertheless, it will be understood that various modifications are made without departing from the spirit and scope of the disclosure. Accordingly, other implementations are within the scope of the following claims.

1. A method for tokenizing genetic sequence data, comprising:
obtaining genetic sequence data;
extracting k-mers from the genetic sequence data as a plurality of k-mer anchors;
appending at least one most abundant k-mer target to each k-mer anchor; and
generating tokens based on the appended k-mer anchors and targets.

2. The method of claim 1, wherein extracting k-mers from the genetic sequence data comprises using a Statistically Primary alignment Agnostic Sequence Homing (SPLASH) technique.

3. The method of claim 1, further comprising appending a count for each appended k-mer target to the k-mer anchor.

4. The method of claim 1, wherein generating tokens comprises replacing absent sequences in a sample with a special token.

5. The method of claim 4, wherein the special token is ‘N’.

6. The method of claim 1, further comprising filtering the k-mer anchors based on entropy and effect size thresholds.

7. The method of claim 6, further comprising checking the filtered k-mer anchors against contaminant and positive lookup tables to identify and exclude certain sequences.

8. A system for analyzing nucleic acid sequences, comprising:
a processor; and
a memory storing instructions that, when executed by the processor, cause the system to:
receive genetic sequence data;
extract k-mers from the genetic sequence data as a plurality of k-mer anchors;
append at least one most abundant k-mer target to each k-mer anchor; and
generate tokens based on the appended k-mer anchors and targets.

9. The system of claim 8, wherein extracting k-mers from the genetic sequence data comprises using a Statistically Primary alignment Agnostic Sequence Homing (SPLASH) technique.

10. The system of claim 8, wherein the instructions further cause the system to append a count for each appended k-mer target to the k-mer anchor.

11. The system of claim 8, wherein generating tokens comprises replacing absent sequences in a sample with a special token.

12. The system of claim 11, wherein the special token is ‘N’.

13. The system of claim 8, wherein the instructions further cause the system to filter the k-mer anchors based on entropy and effect size thresholds.

14. The system of claim 13, wherein the instructions further cause the system to check the filtered k-mer anchors against contaminant and positive lookup tables to identify and exclude certain sequences.

15. A non-transitory computer-readable storage medium storing instructions that, when executed by a processor, cause the processor to perform operations comprising:
obtaining genetic sequence data;
extracting k-mers from the genetic sequence data as a plurality of k-mer anchors;
appending at least one most abundant k-mer target to each k-mer anchor; and
generating tokens based on the appended k-mer anchors and targets.

16. The non-transitory computer-readable storage medium of claim 15, wherein extracting k-mers from the genetic sequence data comprises using a Statistically Primary alignment Agnostic Sequence Homing (SPLASH) technique.

17. The non-transitory computer-readable storage medium of claim 15, wherein the operations further comprise appending a count for each appended k-mer target to the k-mer anchor.

18. The non-transitory computer-readable storage medium of claim 15, wherein generating tokens comprises replacing absent sequences in a sample with a special token.

19. The non-transitory computer-readable storage medium of claim 18, wherein the special token is ‘N’.

20. The non-transitory computer-readable storage medium of claim 19, wherein the operations further comprise filtering the k-mer anchors based on entropy and effect size thresholds, and checking the filtered k-mer anchors against contaminant and positive lookup tables to identify and exclude certain sequences.
